(19) United States
(12) Patent Application Publication
(10) Pub. No.: US 2016/0054279 A1
(43) Pub. Date: Feb. 25, 2016

(54) Method for Detecting Second and Thirdhand Smoke

(71) Applicants: Karen Schlosser, Spokane, WA (US); James Schlosser, Spokane, WA (US); Thomas Caferro, Abington, MA (US)

(72) Inventors: Karen Schlosser, Spokane, WA (US); James Schlosser, Spokane, WA (US); Thomas Caferro, Abington, MA (US)

(21) Appl. No.: 14/466,156

(22) Filed: Aug. 22, 2014

(51) Int. Cl. G01N 33/00

(52) U.S. Cl. CPC G01N 33/0036

(57) ABSTRACT

A novel method for testing if smoking has occurred within a space. Smoking is determined to have happened by testing for and confirming the presence of nicotine using a lateral flow test strip. The nicotine may be captured from secondhand smoke by exposing the condensing pad of the lateral flow tester to air within the space prior to activating the test strip. The nicotine may also be captured as part of thirdhand smoke residue on a surface within the space. A prior art commercially available lateral flow test strip used to test for the presence of cotinine within a biological sample, is used in novel ways for detecting for the past presence of nicotine smoke within a space. Improvements to the test apparatus are also disclosed.

### CROSS REFERENCE TO RELATED APPLICATION

There are no related applications.

### STATEMENT REGARDING FEDERALLY SPONSORED R&D

Not related to this application.

### TECHNICAL FIELD

This invention relates to chemical test technologies to detect both airborne secondhand tobacco smoke, and surface-borne thirdhand tobacco smoke.

### BACKGROUND OF THE INVENTION

The health dangers of smoking tobacco are well known. Firsthand smoke is purposefully inhaled by a smoker through well-known devices such as but not limited to, pipes, cigarettes and cigars. In addition to the user's desired nicotine from smoking, other chemicals, including but not limited to heavy metals and poisons, are also brought into the body. Some users enjoy smoking and don't want to quit, while many others continue to succumb to the addictive nature of nicotine by smoking when they wish not to. These users often feel ashamed and fear others knowing they smoke. As a result many smokers often use hidden locations such as bathrooms, hotel rooms and cars for smoking.

The dangers of second hand smoke are also fairly well understood by the general public. Very much similar to smoking firsthand, second hand smoke causes someone not smoking to also inhale potentially toxic chemicals. The unfortunate thing about second hand smoke is that the person receiving the damage to their body has no desire for nicotine and the chemicals that come with smoking. Public awareness of the dangers of second hand smoke has swung the voters in many states to pass legislation restricting smoking in public locations. Many states force smokers to move outdoors and a certain distance from building entrances. These laws protect the non-smoking public from second hand smoke and further force smokers to undesirable locations. For many smokers, they are even further habitualized to smoke in locations away from public visibility.

Unlike firsthand and secondhand smoke, the dangers of thirdhand smoke is not well known by the general public. Thirdhand smoke is the residue that remains on surfaces and objects as a result of smoking. Long after a smoker finishes a cigarette, a surface may include deposits of nicotine and the toxic chemicals that come along with cigarette smoke. Even worse, residue from the smoke can react with other environmental substances creating even more dangerous substances on surfaces. Thirdhand smoke residue, for example, can be in contact with cooking surfaces getting into food, or on bathroom counters getting into peoples' eyes and mouths. Thirdhand smoke is likely to be the greatest risk to kids and infants as they spend a significant amount of time in contact with floors and living surfaces. Kids and infants are likely to make oral contact with surfaces and are less likely to wash their hands prior to eating or touching other surfaces of their body.

In addition to the damages of smoke on the body, smoke also damages property. With the high level of public awareness on the dangers of smoking, smoked in dwellings and objects generally have less resell value than pristine versions. Business owners that rent spaces to the general public can very much be negatively financially impacted by smokers. Rental owners often require renters to sign a contract that they will not smoke in the owner's rental property. Car rental companies and hotels make customers sign contracts and install reminder signs not to smoke. Many businesses use fines as deterrents.

With smokers increasingly being forced to smoke in hidden non-public places to maintain their addictions and with many smokers being ashamed of smoking, the problem of enforcing non-smoking contracts is very challenging. To catch a smoker in the act of smoking, the business owner either has to invade the smoker's privacy or use expensive electronic smoke detectors. Without a low cost method for detecting thirdhand smoke available today, owners are left with the subjective method of smelling thirdhand smoke. With smokers not wanting to get publicly humiliated for smoking and having an addiction overriding their better judgment, many smokers will lie about smoking. Even without lying, many long term smokers are desensitized from chronic exposure, and are unable to smell smoke and distinguish thirdhand contaminated spaces from pristine ones. Accusations from business owners wanting to protect their investments leads to relationship and business damaging disagreements with customers. Often the cost of losing a customer is more expensive than the immediate smoke damage, so the smell is covered up with fragrances and such. At some point, the repeated cover-ups no longer work which impacts non-smoking customers and property values. There is a need for business owners to protect their property without invading a customer's privacy, damaging the relationship with the customer by relying on subjective evidence, or having to spend significant amounts of money on highly scientific forensic analysis of evidence. For business owners, the goal is to change habits with credible data and not to take customers to court.

The problem of thirdhand smoke and the situation described above is not limited to business situations. It is not uncommon for kids to give into peer pressure and try smoking. Quickly they can become addicted. Similar to the situation previously described, parents will try to keep kids from smoking and kids will try to hide it. There is a need for a parent to test, or audit, if a child is smoking without damaging their relationship with subjective evidence or by forcing biological testing.

It should be appreciated that there are methods today that can determine if firsthand, secondhand or thirdhand smoking contamination has occurred, or is occurring within a space.

Firsthand smoking can be determined though video surveillance. Surveillance equipment is not only expensive, but it may be illegal in some situations. Even if legal, customers will be unlikely to sign a rental contract allowing an invasion of their personal privacy. Biological testing, that is testing urine and blood for cotinine, a byproduct of nicotine, is not a solution. Testing if someone has historically smoked by testing their blood or urine for cotinine does not provide a business owner the data to know if the customer has smoked in a rented space.

Identifying secondhand smoke in a space can be done through the use of gas chromatography/mass spectrometry, as well as highly sensitive and expensive electronic smoke detectors. Air sampling and subsequent gas chromatography/mass spectrometry testing can provide very accurate results. This method of testing is slow and expensive, more suitable for forensic testing. Lower cost common commercial smoke detectors lack the sensitivity and selectivity needed for detecting secondhand cigarette smoke. In addition, smoke detectors are easily disabled.

Currently, thirdhand smoke detection is limited to expensive and slow methods. A wipe of a surface and subsequent chromatographic or mass spectrometric testing of that wipe can provide accurate results, but the process is slow and expensive. Nicotine sensing films comprising conductive polymers linked with a reporting layer can record changes in chemiresistance due to absorption of nicotine, but to date the technologies are cost-prohibitive.

In these respects, the device and methods for detecting secondhand and thirdhand smoke substantially depart from conventional concepts of the prior art, and in doing so provide an economical and quick method for testing for the presence of secondhand and thirdhand smoke in a space.

### SUMMARY OF THE INVENTION

The present invention therefore is directed at providing a test method for determining if smoking has occurred in a space. Smoking can be determined by the presence of secondhand smoke in the air or thirdhand smoke deposits on surfaces. The present invention uses lateral flow test apparatuses for economically and quickly absorbing smoke deposits and flowing them through a test structure using capillary action. The present invention also provides improvements to existing lateral flow cotinine test apparatuses by increasing their sensitivity above what is needed for cotinine testing of biological samples (e.g. blood, urine, saliva). There currently is not an economical, quick, or reliable method for determining if smoking has occurred in a space.

One embodiment of the present invention collects nicotine via ambient contact with airborne secondhand smoke, where the sample collection pad acts as a nicotine condenser. After exposure to ambient secondhand smoke, the lateral flow assay is initiated by introduction of a suitable, often aqueous eluting solvent. The chemical interaction of nicotine with the test strip results in a visual change to the test trap.

In another embodiment of the invention, sample residue is collected from a suspected thirdhand smoke surface via direct swabbing with the sample area of a test stick. The lateral flow assay is then initiated by introduction of a suitable, often aqueous eluting solvent. Alternatively, a suspected thirdhand smoke surface can be wet swabbed with a cotton ball, or similar tool, that has been wetted with a suitable solvent. After swabbing, the lateral flow assay is initiated with the introduction of liquid extracted or squeezed from the swabbing tool. Again, the chemical interaction of nicotine with the test strip results in a visual change of the test trap.

These and other features, aspects, and advantages of the present invention will become better understood with regard to the following description, appended claims, and accompanying drawings.

### BRIEF DESCRIPTION OF THE DRAWINGS

Preferred embodiments of the invention are described below with the reference to the following accompanying drawings:

FIG. 1 is a front perspective view of a test apparatus according to the present invention where a moistened swab is used to gather smoke residue for testing;

FIG. 2 is a front perspective view of an alternative embodiment according to the present invention, wherein a simplified test strip is used to directly swab a surface;

FIG. 3 is a perspective view of a test unit for collecting test particles from air prior to being activated with liquid;

FIG. 4 is perspective view of an alternative embodiment test strip where anti-radial chromatography is used to increase the sensitivity of the test.

### DETAILED DESCRIPTION OF THE PREFERRED EMBODIMENTS

Many of the fastening, connection, manufacturing and other means and components utilized in this invention are widely known and used in the field of the invention are described, and their exact nature or type is not necessary for a person of ordinary skill in the art or science to understand the invention; therefore they will not be discussed in detail. Furthermore, the various components shown or described herein for any specific application of this invention can be varied or altered and anticipated by this invention and the practice of a specific application or embodiment of any element may already be widely known or used in the art, or persons skilled in the art or science; therefore, each will not be discussed in significant detail.

The term “smoke” as used herein is intended to include any type of airborne byproduct created by a user smoking a device. That device may be, but is not limited to, a pipe, cigarette, or an electronic cigarette. The product creating the smoke is generally referred herein to tobacco smoke containing the drug nicotine, but the present invention should not be construed to be limited to tobacco smoke. The present invention applies to other types of substances that are smoked.

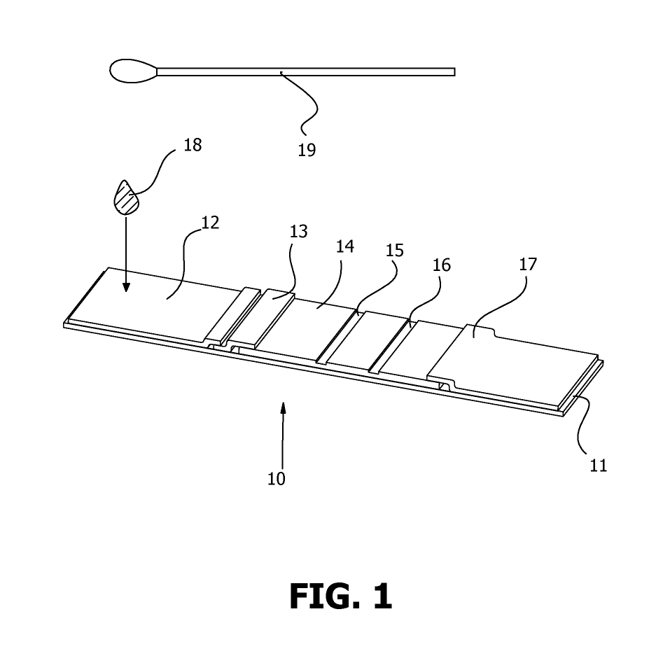

Now referring to drawings. FIG. 1 shows a preferred embodiment of the present invention. A preferred test device assembly 10 is a lateral flow test strip. Although pregnancy test kits are well known by the general public, few know how lateral flow testing technology works. Present day lateral flow cotinine test strips are designed and used to detect cotinine in a person's blood, saliva, or urine. Cotinine, the body's metabolite of nicotine, reacts with a test strip to show a person has recently used a nicotine product. A positive result from a prior art cotinine test only indicates that a person has consumed nicotine. Prior art test strips reveal neither the route of consumption nor the physical location where said nicotine was consumed. For example, nicotine from smokeless tobacco, or a smoking cessation aid such as a nicotine strip, could have created the cotinine in a person's blood or urine. Moreover, a prior art cotinine test strip does not indicate a person smoked in a given space. Therefore, prior art cotinine test strips used with individual's blood, saliva, and/or urine samples do not accomplish the desired results of the present invention. Prior art cotinine test strips can be purchased from the NyMox Corporation.

In preferred test device assembly 10, a base 11 is used as a support structure for other components and to provide rigidity to the assembly during use. Attached to base 11 is sample pad 12. Sample pad 12 is made from an adsorbent material. Sample pad 12 adsorbs a liquid 18 dispensed from a moistened swab 19. Swab 19 is shown as a common cotton or synthetic swab, but it could be a moistened cotton ball or fabric, or such. The present invention should not be construed to be limited to a particular type of swab 19. Swab 19 provides the means for collecting particles from a test surface 21. Swab 19 is pre-moistened with a suitable, often aqueous solvent. Testing has shown that using water as a solvent in combination with commercially available cotinine lateral flow test strips can indicate the presence of nicotine on surfaces.

Swab 19 dispenses fluid 18 onto sample pad 12 via a squeeze or solvent extraction of the tip of swab 19, or by direct contact of the two. By pre-moistening swab 19, its tip more readily captures surface contaminants from surface 21 over that of a dry swab tip. The moisture of swab 19 provides the means for capillary action of lateral flow of test device assembly 10.

Once fluid 18 is dispensed onto sample pad 12, capillary action takes fluid 18 towards a conjugate pad 13 which is connected to sample pad 12 and base 11. Conjugate pad 13 contains-a colored or otherwise detectable antibody-label conjugate which binds with high affinity and selectivity to nicotine. Such conjugates maybe composed of, but are not limited to, antibody receptors deposited on gold nanoparticles. An optional control antibody, commonly Immunoglobulin G or other suitable protein complex, if present, also reacts with fluid 18. Fluid 18 then becomes a mixture containing the original liquid of the swab, the test antibody, potentially a nicotine residue from surface 21, and optionally the control antibody. Conjugate pad 13 provides the means for mixing test fluid 18 with the test antibody. The presence of nicotine analyte in a sample 18 results in irreversible formation of a nicotine-antibody-label ternary construct. This ternary construct is either immediately visually or photometrically detectable, or, more commonly, it travels along a porous medium 14 to where it can be visually or photometrically detected in a trap area 15.

Alternatively, the antibody can reside in trap area 15, where the binding of nicotine analyte prevents the absorption of colored particles, resulting in a colorless positive test. Alternatively, the trap area can be populated with a release ligand, which only captures the labeled antibody in the absence of nicotine analyte, again resulting in a colorless positive test. In fluid connection with porous medium 14 is a wick pad 17. Wick pad 17 provides the necessary capillary action to ensure proper flow of fluid. A more detailed discussion of the lateral flow test process may be found in U.S. Pat. No. 5,238,652 to Sun et al. and U.S. Pat. No. 6,121,008 to Fitzpatrick and Lenda, which are herein incorporated by this reference.

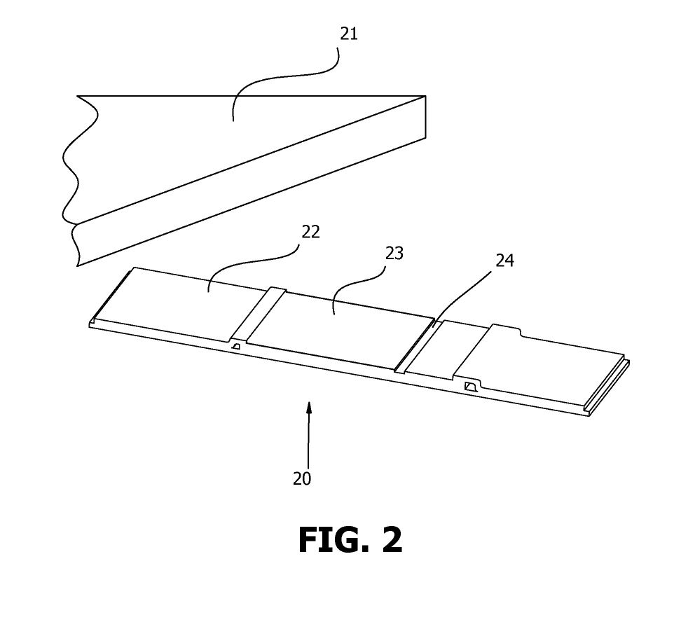

FIG. 2 shows an alternative test strip and can be used to describe a second test method. A simplified test strip 20 is comprised of a sample pad 22 which is in contact and fluid connection with simplified reaction pad 23. Similar to the preferred embodiment according the present invention, simplified test strip 20 uses an antibody for reacting with nicotine molecules to make the lateral flow test process work. Rather than having a second antibody as a control indicator, the alternative embodiment only uses a simplified test trap 24 and not a control trap. The resulting test strip is less expensive to manufacture and provides the desired test results. In this alternative embodiment, simplified sample pad 22 is rubbed against a suspected thirdhand smoke surface, directly capturing potential nicotine residue. The needed liquid for capillary flow of the test strip can be provided to surface 21 prior to contact with simplified pad 22, or the liquid can be applied to simplified pad 22 after it has been rubbed against surface 21. It should be appreciated that the use method of the alternative embodiment of FIG. 2 could also apply to the preferred embodiment of FIG. 1, that is sample pad 11 could be directly applied to surface 21 regardless the addition of a control trap.

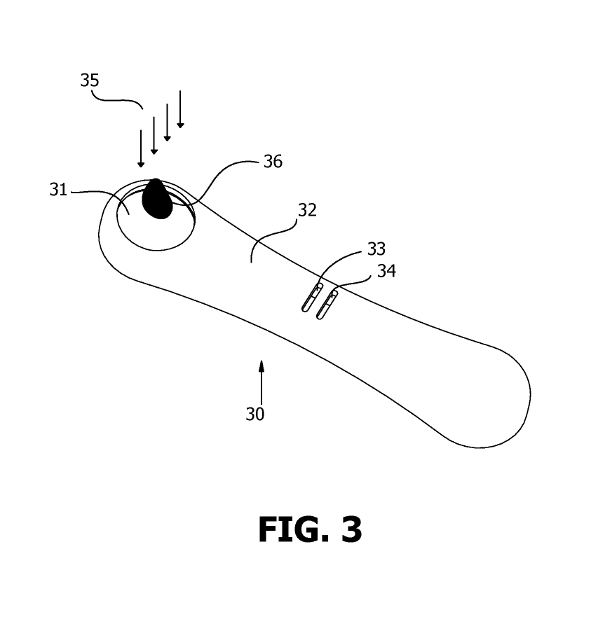

FIG. 3 shows a smoke capturing test strip 30 which is another alternative embodiment of the present invention. This embodiment is similar to the preferred embodiment according to the present invention as it has similar structure to that of FIG. 1. Smoke capturing test strip 30 has a housing 32 for holding the lateral flow test strip. Housing 32 has an optional control trap window 34 and a test trap window 33. A dry smoke capturing pad 31 is in fluid contact with an ambient airflow 35. Smoke capturing test strip 30 is used as a passive smoke capturing device within a space. Consistent with known test design, maximum surface area per unit volume is likely desirable for sample pad 31. Thus, dry smoke capturing pad 31 can be variably configured in a conical, cylindrical, pyramidal, helical, ridged, or otherwise corrugated design such that airborne smoke is efficiently collected. Airflow 35 continually flows over dry smoke capturing pad condenser 31. When desired, a wetting fluid 36 is applied to dry capturing pad 31, activating test strip 30. The resulting test method is then the same as previously described for the preferred embodiment as shown in FIG. 1, wherein via capillary forces a fluid drives a color change of a test trap line. This embodiment can provide more efficient airborne sample collection than can be accomplished with a conventional fluid activated lateral flow test strip.

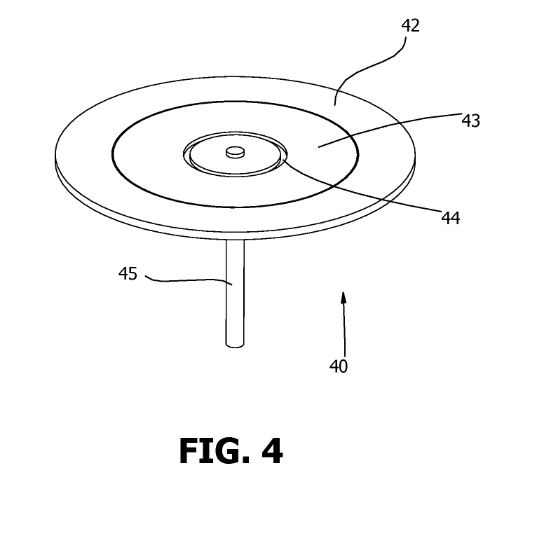

Yet another embodiment of the present invention is an anti-radial flow device 40, shown in FIG. 4. The surface area of an ambient smoke capture area 42 is enhanced relative to the lateral flow stick design. Functioning as a condenser, capture area 42 sensibly benefits from designs with increased surface area, such as those described in the previous section. After exposure to ambient smoke, capture area 42 is uniformly wetted with a suitable, often aqueous solvent. The resulting mixture then travels via capillary action across a membrane 43, toward the center of the anti-radial flow device 40. This capillary flow effectively concentrates the collected nicotine, thereby affording greater sensitivity over standard linear flow cotinine testing. The nicotine is then detected at a circular trap line 44. An absorbent wick 45 ensures proper capillary flow.

Further embodiments are possible without departing from the scope of the present invention. For instance, another embodiment may use the configuration and use of test devices previously described herein, but may include a nicotine hapten derived antibody optimized for testing smoke residuals, rather than cotinine hapten derived antibody optimized for bio-fluid testing, thereby affording greater sensitivity and selectivity to the test strip.

While the device and methods for detecting secondhand and thirdhand smoke herein described constitute preferred embodiments of the invention, it is to be understood that the invention is not limited to these precise form of assemblies, and that changes may be made therein without departing from the scope and spirit of the invention as defined in the appended claims.

1. A method for testing the presence of smoke deposits on a surface comprising the steps of:
(a) applying a moistened swab to a surface having a suspected smoke deposit, said moistened swab and said suspected smoke deposit forming a test fluid;
(b) dispensing said test fluid to an adsorbent condenser fluidly connected to a conjugate pad, said conjugate pad containing a smoke test antibody, said test fluid and said smoke test antibody mixing to form a test mixture;
(d) said test mixture flowing via capillary forces to a test trap area wherein said smoke test antibody and said smoke deposit reacts with said test trap area to create a test trap line indicating the presence of said smoke deposit on said surface.

2. The method for testing for the presence of smoke deposits on a surface as recited in claim 1, where a control antibody mixes with said test fluid and said control antibody provides a visible line on a control trap area.

3. A method for testing the presence of nicotine smoke comprising the steps of:
(a) contaminating an air sampling pad with a smoke deposit by subjecting said air sampling pad to an amount of air;
(b) applying a fluid to said air sampling pad;
(b) said fluid and said smoke deposit flowing to a conjugate pad via capillary forces;
(c) said conjugate pad mixing a test antibody with said fluid and said smoke deposit to form a test mixture; and,
(d) said text mixture flowing via capillary forces to a test trap area wherein said test antibody and said smoke deposit reacts with said test trap area to create a test trap line indicating the presence of said smoke deposit in said amount of air.

4. The method for testing for the presence of a smoke deposit within an amount of air as recited in claim 3, where a control antibody mixes with said fluid and said control antibody provides a visible line on a control trap area.

5. A method for testing the presence of smoke deposits on a surface comprising the steps of:
(a) rubbing an adsorbent test pad on a potentially contaminated surface having a nicotine residue;
(b) applying an activator liquid to said adsorbent test pad;
(c) said activator liquid carrying said nicotine residue to a conjugate pad via capillary forces;
(d) a test antibody on said conjugate pad reacts with said nicotine residue within said activator liquid;
(e) said activator liquid carrying said nicotine residue and said test antibody to a test trap via capillary forces;
(f) said test trap changes color indicating the presence of said nicotine on said potentially contaminated surface.
